(19) United States
(12) Patent Application Publication
(10) Pub. No.: US 2024/0215967 A1
(43) Pub. Date: Jul. 4, 2024

(54) BIOABSORBABLE OCCLUSION SYSTEM

(71) Applicant: Teleflex Life Sciences III LLC, Wilmington, DE (US)

(72) Inventors: Joshua Brenizer, Oak Grove, MN (US); Darren Prom, Coon Rapids, MN (US); Christopher E. Buller, Toronto (CA); Dean Peterson, Minneapolis, MN (US)

(73) Assignee: Teleflex Life Sciences III LLC, Wilmington, DE (US)

(21) Appl. No.: 18/393,787

(22) Filed: Dec. 22, 2023

Related U.S. Application Data
(60) Provisional application No. 63/478,286, filed on Jan. 3, 2023.

(51) Int. Cl. A61B 17/00

(52) U.S. Cl. CPC A61B 17/0057; A61B 2017/00004; A61B 2017/00606; A61B 2017/00641; A61B 2017/00654; A61B 2017/00676

(57) ABSTRACT

An embodiment of the present disclosure a bioabsorbable occlusion system for occluding defects or openings in a heart.

### CROSS-REFERENCE TO RELATED APPLICATIONS

This application claims the benefit of priority under 35 U.S.C. § 119(c) to U.S. Provisional Appln. Ser. No. 63/478,286, filed Jan. 3, 2023, which is incorporated herein by reference in its entirety.

### TECHNICAL FIELD

The present disclosure relates to a bioabsorbable occlusion system for openings or defects in a heart.

### BACKGROUND

Openings and defects in a heart are often treated by placing an occlusion device at the site of the opening. Conventional occlusion devices, e.g., a Amplatzer Septal Occluders, for heart defects are metallic braided structures. These devices have a pre-defined shape and are collapsed inside a delivery tube for insertion into the heart and expand upon exit of the delivery tube to occlude the opening. Such conventional occlusion devices are designed to be permanently implanted in place in the heart and do not absorb or biodegrade into the surrounding tissue. Long term implantation may lead to cardiac erosion in some cases. In addition, septal closure with permanent implant can preclude septal crossing for future interventions

### SUMMARY

An embodiment of the present disclosure is a bioabsorbable occlusion system for an opening or defect in a wall of a heart. The bioabsorbable occlusion system includes an occlusion implant having a first anchor, a second anchor, and a connector element coupled to the first anchor and the second anchor and configured to draw the first and second anchors toward each other. In such a system, an entirety of the occlusion implant is bioabsorbable. In addition, the occlusion implant has an insertion configuration, where the first anchor and the second anchor are collapsed and have an initial cross-sectional dimension, and an expanded configuration, where the first anchor and the second anchor are expanded outwardly and have expanded cross-sectional dimension that is substantially greater than the initial cross-sectional dimension.

Another embodiment of the present disclosure is a method for occluding an opening or a defect in a heart. The method includes inserting a guide catheter over a guidewire so that its distal end is located proximate the opening or defect in the tissue of the heart. The method includes advancing a delivery device in a distal direction through a channel of the guide catheter so that a first anchor and a connector element of an occlusion implant is positioned adjacent to a first side of the tissue of the heart and the opening or defect. In such a method, an entirety of the occlusion implant is bioabsorbable. The method includes further advancing the delivery device in the distal direction through the channel of the guide catheter so that a second anchor of the occlusion implant is positioned adjacent to a second side of the tissue of the heart and overlies the opening or defect. Next, the method includes retracting the connector element to draw the first anchor toward the second anchor to secure the occlusion implant in place. Then, the occlusion implant will be bioabsorb. The method may be used for a number of different openings or defects, which include, but are not limited to patent foramen ovale, ductus arteriosis, an iatrogenic transcaval defect, a ventricular septal defect, or an arterio-venous fistulae.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the present application, there is shown in the drawings illustrative embodiments of the disclosure. It should be understood, however, that the application is not limited to the precise arrangements and instrumentalities shown. In the drawings:

FIG. 1 is a schematic cross-sectional view of a heart with a bioabsorbable occlusion implant shown occluding an opening, according to an embodiment of the present disclosure;

FIG. 2A is a side view of the bioabsorbable occlusion implant shown in FIG. 1 in an collapsed, insertion configuration with the delivery assembly removed for clarity of illustration;

FIG. 2B is a detailed sectional side view of the bioabsorbable occlusion implant shown in FIG. 1 in an expanded and implanted configuration;

FIG. 3 is a plan view of a first anchor of the occlusion implant shown in FIG. 2 according to an embodiment of the present disclosure;

FIG. 4 is a side view of the first anchor of the occlusion implant shown in FIG. 3;

FIG. 5 is a plan view of a second anchor of the occlusion implant shown in FIG. 2 according to an embodiment of the present disclosure;

FIG. 6 is a side view of the first anchor of the occlusion implant shown in FIG. 5;

FIG. 7 is a plan view of a first anchor of the occlusion implant shown in FIG. 2 according to another embodiment of the present disclosure;

FIG. 8 is a side view of the first anchor of the occlusion implant shown in FIG. 7;

FIG. 9 is a plan view of a second anchor of the occlusion implant shown in FIG. 2 according to an embodiment of the present disclosure;

FIG. 10 is a side view of the first anchor of the occlusion implant shown in FIG. 10;

FIGS. 11A-11D illustrates a bioabsorbable occlusion system according to another embodiment of the present disclosure;

FIG. 12 illustrates a bioabsorbable occlusion system carrying the occlusion implant in an insertion configuration;

FIGS. 13A through 14 illustrate a sequence of implanting the occlusion implant shown in FIG. 11 to occlude an opening in a tissue of the heart.

FIG. 15 is a side schematic view of an occlusion implant in an insertion configuration according to another embodiment of the present disclosure;

FIG. 16 is a side schematic view of the occlusion implant shown in FIG. 15, shown in an expanded configuration; and

FIGS. 17 through 19 illustrate a sequence of implanting the occlusion implant shown in FIGS. 15 and 16, to occlude an opening in a tissue of the heart according to an embodiment of the present disclosure.

### DETAILED DESCRIPTION OF ILLUSTRATIVE EMBODIMENTS

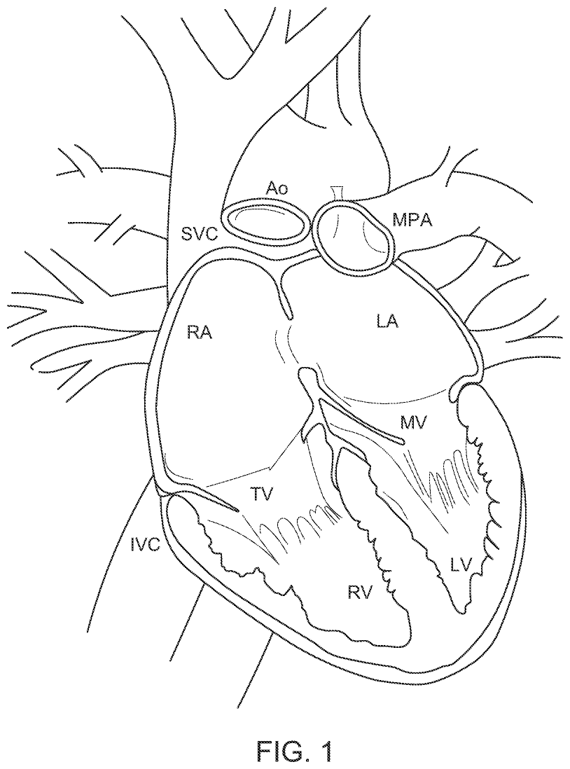

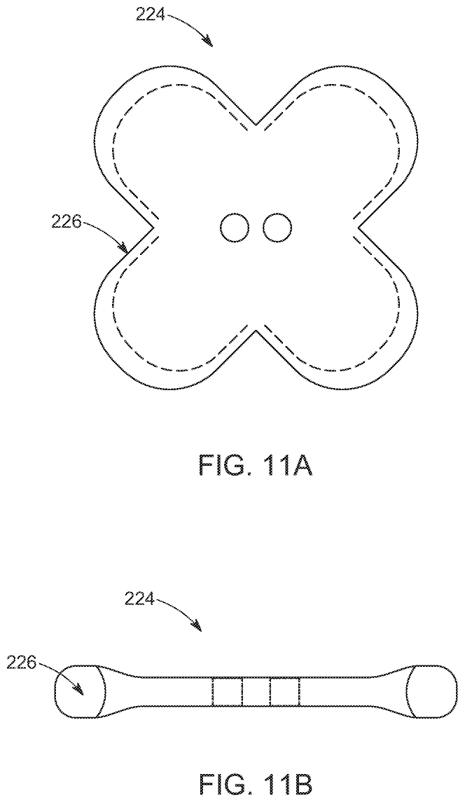

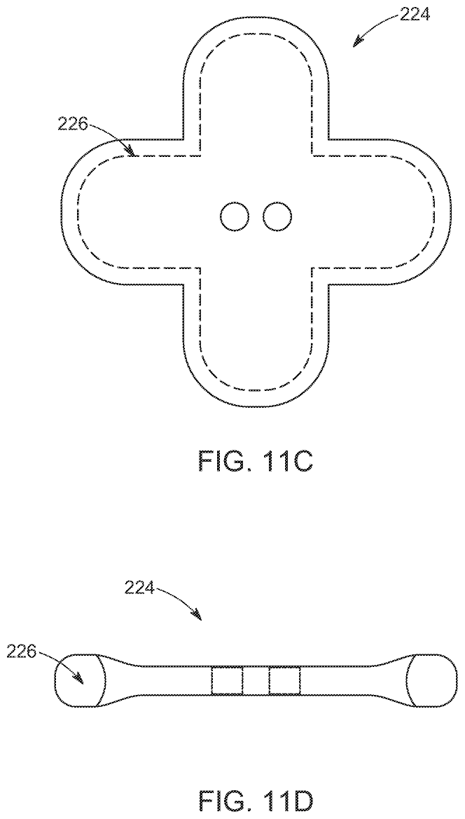

As shown in FIG. 1, embodiments of the present disclosure include a bioabsorbable occlusion system 10 for an opening or defect in a tissue, e.g., a wall, of a heart H. More specifically, the bioabsorbable occlusion system 10 includes an occlusion implant 20 that is configured to occlude defects/openings in the heart H and a delivery assembly 60 (FIG. 11). The occlusion implant 20 is configured to entirely absorb into the patient over a defined time period. For example, the occlusion implant may fully resorb between 6 months and 18 months after implantation. In another example, the occlusion implant may fully absorb between 9 months and 18 months from implantation. In another example, the occlusion implant may fully absorb between 12 months and 18 months from implantation. As used herein, the term “bioabsorbable” means that the material will degrade over time by enzymatic action, by hydrolytic action and/or by other similar mechanisms in the human body, such that the products of biodegradation will either be absorbed by tissue within the body or excreted or through endothelialization, where implant is replaced by human tissue.

Continuing to FIG. 1, an atrial septal defect (ASD) is shown in a schematic of a heart H. The bioabsorbable occlusion implant 20 is implanted in the ASD to occlude flow between the left atrium LA and right atrium RA. While an atrial septal defect ASD is shown in the figures, other indications are suitable for the bioabsorbable occlusion implant 20. For example, the bioabsorbable occlusion implant 20 may be used for several types of openings or defects in the heart, which also include, but are not limited to, patent foramen ovale, ductus arteriosis, an iatrogenic transcaval defect, a ventricular septal defect, and/or an arterio-venous fistulae. Accordingly, the bioabsorbable occlusion implant 20 is not limited for use solely for occluding atrial septal defects.

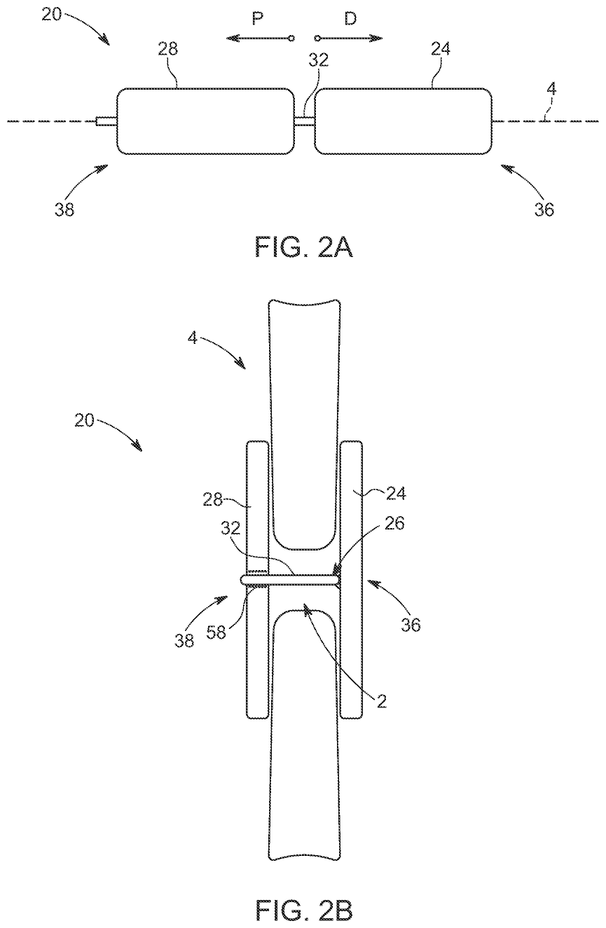

Referring to FIGS. 2A, 2B, and 11, the bioabsorbable occlusion system 10 includes an occlusion implant 20 having a first anchor 24, a second anchor 28, and a connector element 32 coupled to the first anchor 24 and the second anchor 28 and configured to draw the first anchor 24 and the second anchor 28 toward each other. In such a bioabsorable occlusion system 10, an entirety of the occlusion implant 20 is bioabsorbable into the patient.

The occlusion implant 20 has an insertion configuration, as shown in FIGS. 2A and 11, where the first anchor 24 and the second anchor 28 are collapsed and have an initial cross-sectional dimension while being carried by the delivery assembly 60 (FIG. 11).

The occlusion implant 20 also has an expanded configuration, as shown in FIG. 2B where the first anchor 24 and the second anchor 28 are ejected from the delivery assembly 60 and expanded outwardly. FIG. 11 illustrates components of the delivery assembly 60 and is described in more detail further below. In the expanded configuration, the occlusion implant 20 has an expanded cross-sectional dimension that is substantially greater than the initial cross-sectional dimension. Furthermore, the first anchor and the second anchor each have a cross-sectional dimension that is substantially greater than a cross-sectional dimension of the connector element 32. Accordingly, when the occlusion implant 20 is in the expanded configuration, the first anchor expands outwardly to overlies an entirety of a first side of the tissue of the heart around the edge of the opening or defect. Likewise, a second anchor in the expanded configuration overlies an entirety of a second side of the tissue of the heart around the edge of the opening or defect.

Referring to FIGS. 2-6, an embodiment of the first anchor 24 and second anchor 28 is shown. The first anchor 24 and the second anchor 28 are substantially the same. However, as illustrated in the figures, the first anchor 24 may be considered a distal anchor and define a distal end 36 of the occlusion implant 20. The second anchor 28 may be considered a proximal anchor and define a proximal end 38 of the occlusion implant 20. In the insertion configuration as show in FIG. 2A, the occlusion implant 20 may be elongated along an axis A. Furthermore, a distal direction D refers to the direction from proximal end 38 toward the distal end 36 and a proximal direction P refer to the direction from the distal end 36 toward the proximal end 38. In this disclosure, the terms distal anchor and first anchor may be used interchangeably, and the terms proximal anchor and second anchor may be used interchangeably.

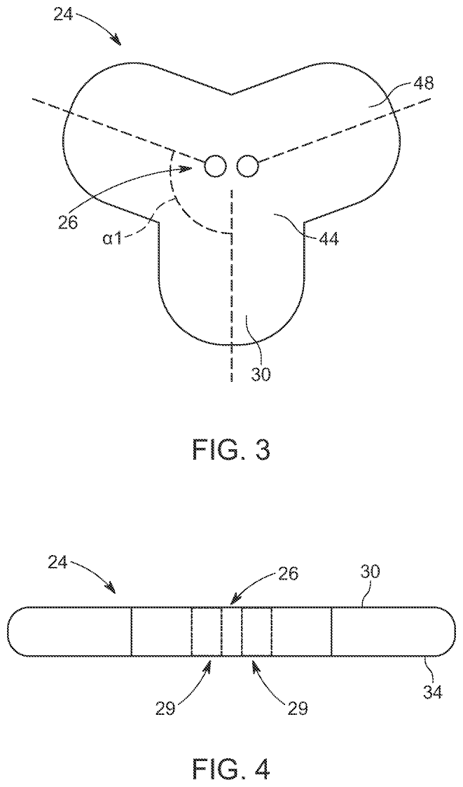

Turning to FIGS. 3 and 4, the first anchor 24 has a size and shape (when in the expanded configuration) that is sufficient cover and overlie a defect. As shown, the first anchor 24 has a central portion 44 and a plurality of lobes 48 that extend radially outwardly from the central portion 44. In the example shown, the first anchor has three lobes. The three lobes 48 may extend outwardly along axes that define an angle α1 between about 110 and about 130 degrees with respect to each other. For example, angle α1 may be about 120 degrees.

Continuing with FIGS. 3 and 4, the first anchor 24 includes a wall facing surface 30 and an opposing surface 34 spaced apart from the wall-facing surface 40. The wall facing surface 30 is configured to face the tissue of the heart H when expanded and implanted to overly the defect 2 (FIG. 2B). The first anchor 24 is configured to be coupled to the connector element 32.

As shown in FIGS. 2B-3, the first anchor 24 includes a coupler 26 disposed along the wall facing surface 30. The coupler 26 connects the connector element 32 to the first anchor 24. The coupler 26 may be any mechanical connector that can be secured to the connector element 32. For instance, the coupler 26 may be two through-bores 29 that extend through the anchor through which a connector element 32 can be looped and knotted, for instance, when the connector element 32 is a suture. In another example, the coupler 26 can be a threaded bore or shaft for threadably coupling to the connector element 32. The coupler 26 may be absorbable. However, in alternative embodiments, the coupler may not be absorbable.

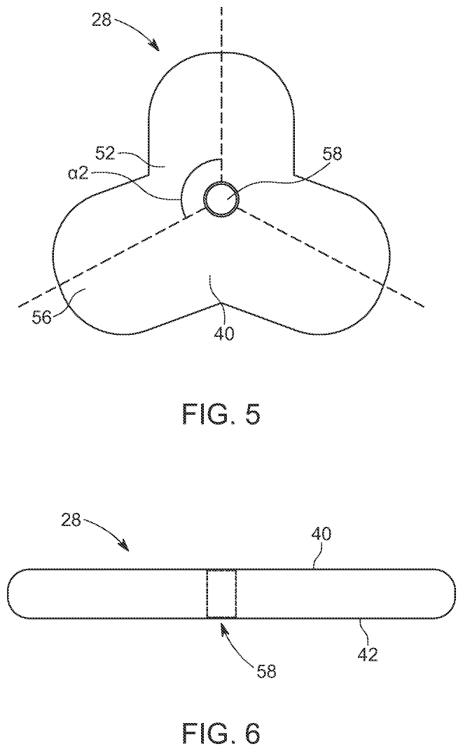

Continuing with FIGS. 5 and 6, the second anchor 28 also has a size and shape (when in the expanded configuration) that is sufficient to cover and overlie a defect 2. As shown, the second anchor 28 has a central portion 52 and a plurality of lobes 56 that extend radially outwardly from the central portion 52. As shown, the second anchor 52 has three lobes 56 that complement and correspond to the lobes 48 of the first anchor 24 (see FIG. 3). Furthermore, the three lobes 56 may extend outwardly along axes that define an angle α2 between about 110 and about 130 degrees with respect to each other. For example, angle α2 may be about 120 degrees.

Continuing with FIGS. 5-6, a central bore 58 extends through the second anchor 28 and sized so that the connector element 32 extends through the bore 58. The second anchor 28 also includes a wall facing surface 40 and an opposing surface 42 spaced apart from the wall-facing surface 40. The wall facing surface 40 is configured to face the tissue of the heart H when expanded and implanted to overlie the defect 2.

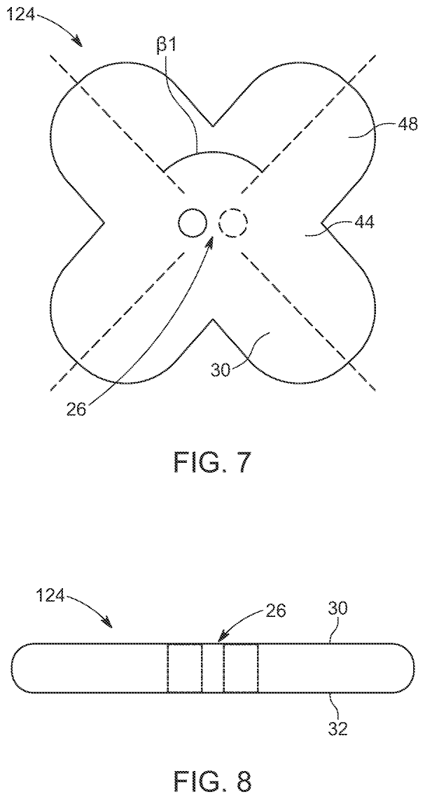

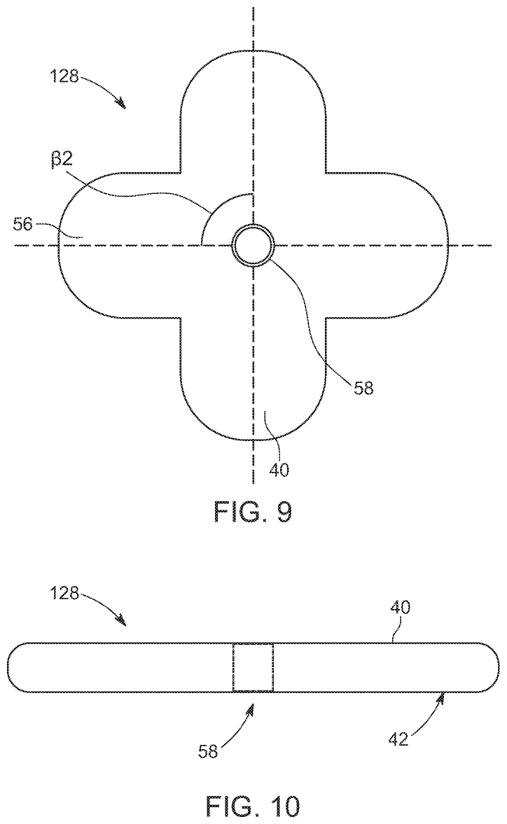

FIGS. 7-10 illustrate an alternative embodiment of the first anchor 124 and the second anchor 128. Features common to anchors 24, 28 and anchors 124, 128 will use like reference numbers. As shown, each of the first anchor 124 and the second anchor 128 have four separate lobes 48, 56 that extend outwardly from their respective central portions. Furthermore, the four lobes may extend outwardly along axes that define an angle β1, β2 between about 80 and about 100 degrees with respect to each other. For example, angle β1, β2 may be about 90 degrees. The first anchor 124 also includes a coupler 26 for connection to the connector element 32 (not shown in FIGS. 7-10) and the second anchor 128 includes a bore 58 through which the connector element 32 may extend.

In yet another embodiment (not shown), each of the first anchor and the second anchor have two outwardly extending lobes and an enlarged central portion. The anchor body itself has a coverage area sufficient to overlie and occlude the target defect.

In yet another embodiment (not shown), each of the first anchor and the second anchor may be a substantially disc shaped body having a coverage area sufficient to overlie and occlude the target defect. In such an embodiment, the first and second anchors may have a generally circular perimeter, an ovular perimeter, or a rectilinear perimeter.

In yet another embodiment (not shown), each of the the first anchor and the second anchor are formed from a frame of separate anchor members that are entirely absorbable. In such an embodiment, the frame of anchor members may be braided materials or other frame like structures with shape memory such that, when the anchors are ejected from the delivery assembly 60, the anchors expanded outwardly. The anchor member may be made from bioabsorable polymers as described herein.

FIGS. 11A-11D illustrate yet another embodiment of the present disclosure. In the embodiment shown, each anchor 224 may have an inflatable edge portion 226 that extends around the anchor outer perimeter. The inflatable edge portion 226 may be defined by an internal channel or bladder like member that extends around the outer perimeter. An injection solution, such as saline, contrast, and the like, may be injected into the internal channel to cause edge expansion as described. In this manner, the outer edge of the first anchor is at least partially inflatable to define its expanded shape. Likewise, the outer edge of the second anchor is at least partially inflatable to define its expanded shape. In such an embodiment, the first and second anchor may include an injection element coupled to first and second anchors and controllable via an actuator at the proximal end of the delivery assembly. The embodiment shown in FIGS. 11A-11D may be similar structure and features to the embodiment shown in FIGS. 1-10.

The first anchor and the second anchor may be arranged on the connector element such that their shapes complement each other to maximize the surface contact with the tissue of the heart H. For example, in embodiments where each anchor has a plurality of lobes, the first anchor is offset with the respect to second anchor so that their respective lobes do not overly along a common axis.

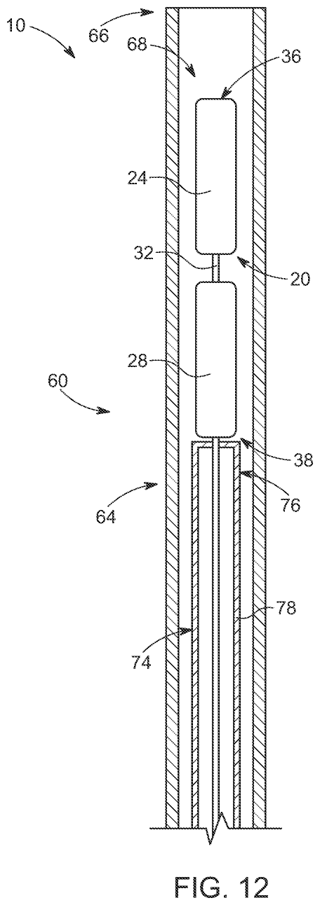

Referring to FIGS. 2 and 12, the connector element 32 may be any absorbable device or element that can be used to couple the first and second anchor together. In the example shown, the connector element 32 may be an absorbable suture that is coupled to and knotted to the first anchor at the coupler 26. Such a suture can extend proximally from the coupler 26 and through the bore 58 of the second anchor 28. In one example, a slip knot or other knot configuration can be used to secure the first and second anchors relative to each other. That is, once the first anchor is implanted and in place, and the second anchor is implanted and in place, tension applied to the suture (or connector element) can draw the two anchors together, at which point a knot or the like can be used to secure the positions of the anchors along the suture, thereby keeping the occlusion implant 20 in place. In an alternative embodiment, the occlusion implant 20 may include an optional lock, in the form of slidable and crimped element can be sliding secured in place along the connector element.

In another embodiment (not illustrated), the connector element 32 may include surface with a plurality of teeth while a lock element can include a plurality of teeth, that when engaged, allow move of the lock distally but inhibit movement of the lock proximally. In addition, the lock element in such an example may be formed directly in the bore 58 of the second anchor and the connector element may be integrally formed with first anchor 24. Thus, in one example, the connector element includes a plurality of teeth that are configured to engage a plurality of teeth located in a bore of the first anchor and the second anchor.

In yet another embodiment (not illustrated), the connector element 32 may be an elongated shaft that is integrally coupled to the first anchor 24. The elongated shaft may include any number projections (or recesses) that are configured to couple to a lock element that includes corresponding recesses (or projections) that when engaged with the elongated shaft serve to fix the position of the first and second anchor relative to each other. In addition, the lock element in such an example may also be formed directly in the bore 58 of the second anchor 28. In such an example, the elongate shaft of the connector element has a plurality of engagement members that are configured to engage to a plurality of corresponding engagement members in the bore of the first anchor or the second anchor.

The first anchor, second anchor, and connector element may be formed from biocompatible and bioabsorbable, polymers, copolymers, or polymer mixtures. Exemplary bioabsorbable materials include polymers and co-polymers of glycolide, lactide, caprolactone, trimethylene carbonate, dioxanone, and physical and chemical combinations thereof. Other examples include polylactic acid (PLA), polyglocolic acid (PGA), poly lactide-co-glycolide (PLGA), among others. In addition, metal alloys that are bioabsorbable may be used if sufficient degradation is possible for a given time.

In certain examples, the first and second anchors may be made of braided and/or woven textile structures that can 1) retain an expanded shape once ejected from the delivery assembly, and 2) bioabsorb over time. For example, the anchors may be braided constructs formed from heat set-able and bioabsorable polymer yarns and/or filaments that are formed into the expanded shape. Such materials can be collapsed for insertion into the delivery assembly for use.

In an example, each component of the occlusion implant 20 is formed from similar polymeric materials so that each component has a somewhat similar degradation profile. However, in certain cases, the material make-up of each component, such as between the anchors 24, 28 and the connector element 32, may differ such that an entirety of the implant can bioabsorb at substantially the same rate. For example, the first anchor and the second anchor may be formed from a first polymeric material and the connector element may be formed from a second polymeric material that is different from the first polymeric material. In another example, the first anchor may be formed from a first polymeric material, the connector element may be formed from a second polymer material, and the second anchor may be formed from a third polymeric material where the first, second, and third polymeric materials are different.

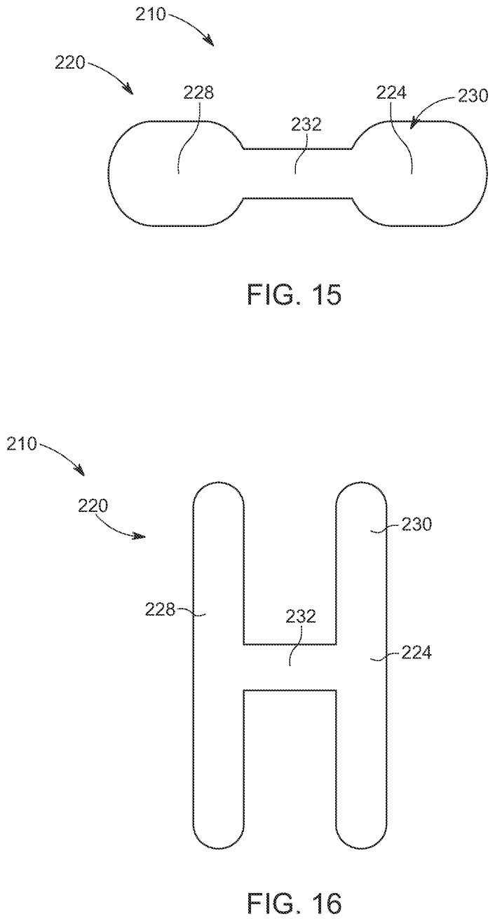

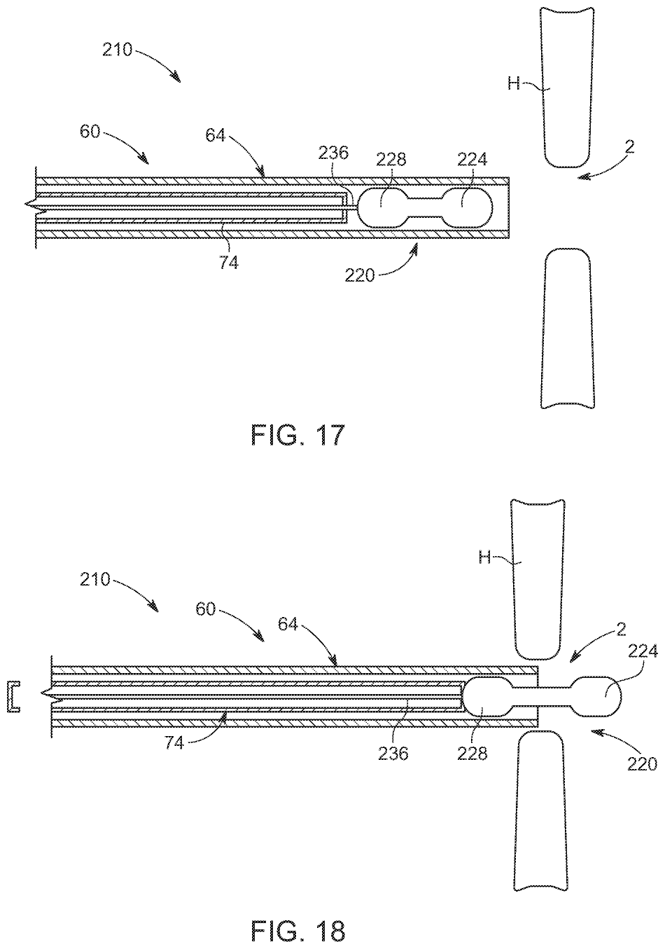

In addition, as further described below, the occlusion implant may be formed from an inflatable polymeric membrane (FIGS. 15-19) that has a pre-defined shape and is subject to degradation in accordance with the teaching as disclosed in the present application.

Referring to FIGS. 12-14, bioabsorbable occlusion system 10 includes a delivery assembly 60 for inserting and implanting the occlusion implant 20. The delivery assembly 60 includes a guide catheter 64, a delivery device 74, and an optional access sheath (not shown or numbered).

As shown in FIG. 12, the guide catheter 64 includes a proximal end (not shown or numbered), a distal end 66 opposite the proximal end), and a channel 68 that extends therethrough. The guide catheter 64 carries occlusion implant 20 in the insertion configuration.

The delivery device 74 is movable within and relative to the guide catheter. As shown, the delivery device 74 is configured to advance the occlusion implant 20 from inside the channel 68 to a location outside the channel 68 (or distal to the distal end 66). The delivery device 74 includes an elongated shaft 78 with a proximal end (not shown or numbered) and a distal end 76 opposite the proximal end. In one example, the delivery device 74 is an elongated push rod. In another example, the delivery device 74 is an elongated tube having a channel for receiving a portion of the connector element therein. Furthermore, the delivery device may be configured to be releasably coupled to the occlusion implant 20.

Continuing with FIG. 12, the occlusion implant 20 may be carried by the delivery assembly 60 for insertion into the defect 2. More specifically, the second anchor 28, which is referred to sometimes as the proximal anchor, is in the guide catheter 64 at the distal end 76 of the delivery device 74. In one example, the proximal anchor or second anchor is in contact with but necessarily coupled to the delivery device 74. In another example, the distal end of the delivery device 74 is coupled to the second anchor 28. The connector element 32 may extend through (or along-side) the second anchor 28 and further into the channel of the guide catheter. The first anchor 24, sometimes referred to as the distal anchor is positioned in the guide catheter adjacent to the second anchor 24. In this manner, the occlusion implant 20 is disposed in the insertion configuration in a generally elongated state. Furthermore, the first anchor 24 and second anchor 28 are formed in a pre-defined shape and are carried by the guide catheter so that upon ejection from the guide catheter, the first and second anchors can be expanded into the expanded configuration.

In typical procedure for occluding defects, the bioabsorable occlusion system 10 may include an access sheath (not shown). The access sheath can have a proximal end, a distal end, and an access sheath channel that extends from the proximal end to the distal end. The delivery assembly 60, e.g., the guide catheter, is insertable into the access sheath channel.

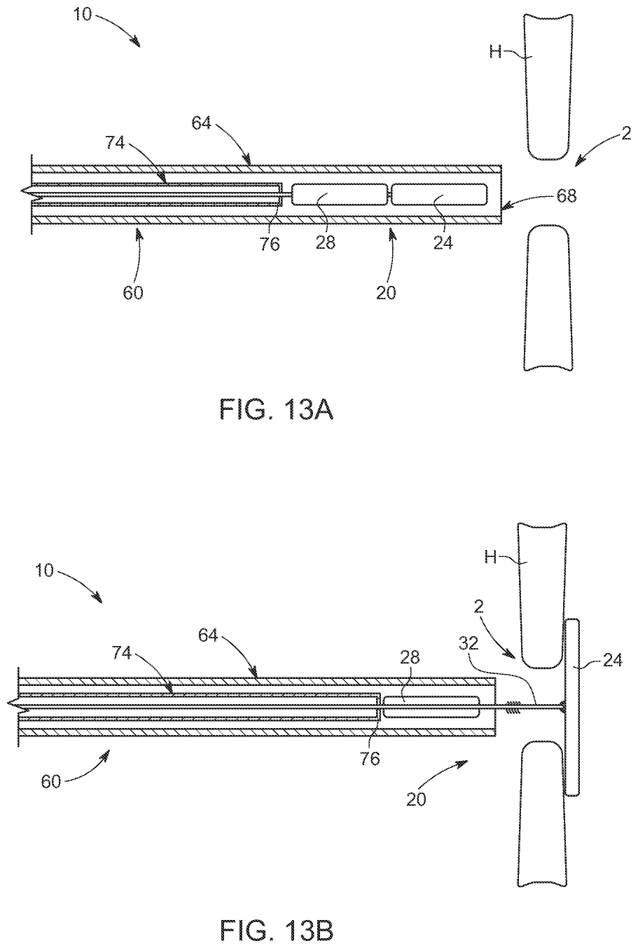

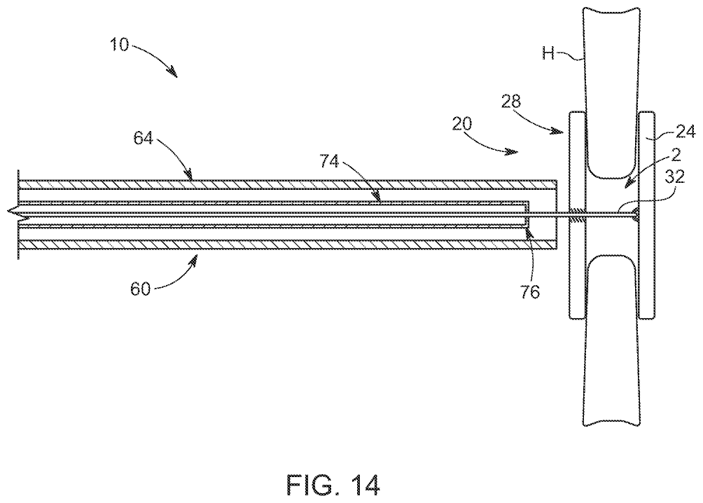

As shown in the sequence of FIGS. 13A-14, advancement of the delivery device 74 in a distal direction ejects the first anchor 24 from the guide catheter 64, thereby causing the first anchor 24 to expand into the expanded configuration. Further advancement of the delivery device 74 in the distal direction ejects the second anchor 28 from the guide catheter, thereby causing the second anchor 28 to expand into the expanded configuration. While advancement of the delivery device 74 is described as causing ejection of the anchors 24, 28, retraction of the guide catheter relative to the delivery device 74 could also cause ejection of the anchors, 24, 28 in the sequence shown.

An alternative embodiment of a bioabsorable occlusion system 210 is shown in FIGS. 15-19. The bioabsorbable occlusion system 210 is similar to the bioabsorable occlusion system 10 described above and shown in FIGS. 2A-14 and common reference numbers are used to identify features common to both systems.

Continuing with FIGS. 15-19, the bioabsorable occlusion system 210 includes an occlusion implant 220 having a first anchor 224, a second anchor 228, and a connector element 232 coupled to the first anchor 224 and the second anchor 228 that couples and second anchors 224, 228 together. In the bioabsorable occlusion system 210, an entirety of the occlusion implant 220 is bioabsorbable into the patient. However, occlusion implant 220 generally includes an expandable body formed from a flexible membrane 230 that defines an inner volume (not shown) configured to receive therein a fluid that causes expansion of the membrane 230 and thus the implant. The flexible membrane 230 may be a woven structure with an impermeable coating, a braided structure with an impermeable coating, or any polymeric material or composite, with can mono-layered or multi-layered, which can contain a fluid to but is flexible enough to permit expansion.

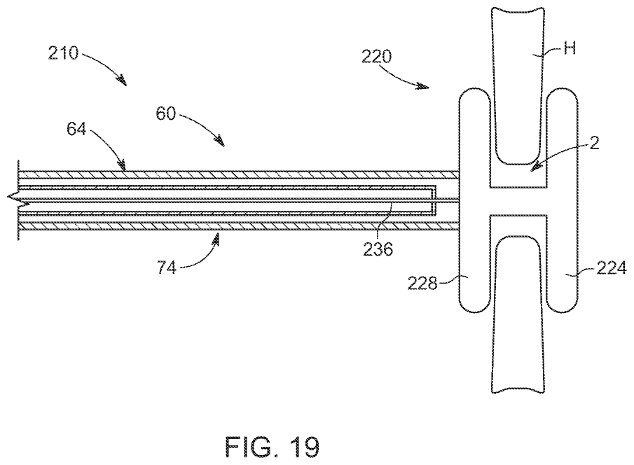

Like occlusion implant 20, the occlusion implant 220 has an insertion configuration, as shown in FIG. 15, where the first anchor 24 and the second anchor 28 are collapsed and have an initial cross-sectional dimension while be carried by the delivery assembly 60 (FIG. 16). The occlusion implant 220 also has an expanded configuration, as shown in FIG. 16, where the first anchor 24 and the second anchor 28 are ejected from the delivery assembly 60 (FIGS. 16 and 19) and expand outwardly. In the expanded configuration, the first anchor 224 and second anchor 228 have an expanded cross-sectional dimension that is substantially greater than the initial cross-sectional dimension. In addition, the cross-sectional dimension of the connector member 232 is substantially less the respective cross-sectional dimensions of the first anchor 224 and the second anchor 228.

The occlusion implant 220 also includes an injection member 236 that is used to inject a fluid into to inner volume of the occlusion implant 220. A fluid may be saline, a contrast, or methyl-acrylate. Other injectable materials that are biocompatible may be used to cause expansion of the implant. In addition, the entirety of the occlusion implant 220 is bioabsorable.

As shown in the sequence of FIGS. 16-19, advancement of the delivery device 74 in a distal direction ejects the first anchor 224 from the guide catheter 64. Further advancement of the delivery device 74 in the distal direction ejects the second anchor 228 from the guide catheter. While advancement of the delivery device 74 is described as causing ejection of the anchors 24, 28, retraction of the guide catheter relative to the delivery device 74 could also cause ejection of the anchors, 24, 28 in the sequence shown. Next, an injection fluid is inserted into the inner volume of the occlusion implant 220 via the injection member 236 until the implant attains it fully expanded state, as shown in FIGS. 16 and 19.

While the disclosure is described herein, using a limited number of embodiments, these specific embodiments are not intended to limit the scope of the disclosure as otherwise described and claimed herein. The precise arrangement of various elements and order of the steps of articles and methods described herein are not to be considered limiting. For instance, although the steps of the methods are described with reference to sequential series of reference signs and progression of the blocks in the figures, the method can be implemented in an order as desired.

1. A bioabsorbable occlusion system for an opening or defect in a wall of a heart, comprising:
an occlusion implant having a first anchor, a second anchor, and a connector element coupled to the first anchor and the second anchor and configured to draw the first and second anchors toward each other, and an entirety of the occlusion implant is substantially bioabsorbable,
wherein the occlusion implant has an insertion configuration, where the first anchor and the second anchor are collapsed and have an initial cross-sectional dimension, and an expanded configuration, where the first anchor and the second anchor are expanded outwardly and have expanded cross-sectional dimension that is greater than the initial cross-sectional dimension.

2. The bioabsorbable occlusion system according to claim 1, wherein the first anchor includes a coupling element that attached the connector element to the first anchor, and wherein the second anchor includes at least one opening through which the connector element extends.

3. The bioabsorbable occlusion system according to claim 1, further comprising a lock member located on the connector element, wherein the lock member is configured to fix the second anchor in position along the connector element relative to the first anchor, thereby drawing the first anchor and the second anchor toward each other.

4. The bioabsorbable occlusion system according to claim 1, wherein the first anchor has a central portion and a plurality of lobes that extend radially outwardly from the central portion, and the second anchor has a central portion with a plurality of lobes that extend radially outwardly from the central portion.

5. The bioabsorbable occlusion system according to claim 4, wherein the first and second anchors each have at least three lobes.

6. The bioabsorbable occlusion system according to claim 4, wherein three lobes extend outwardly along axes that are between 110 and 130 degrees with respect to each other.

7. The bioabsorbable occlusion system according to claim 4, wherein the first and second anchors have four lobes that extend outwardly along axes that are between 80 and 100 degrees with respect to each other.

8. The bioabsorbable occlusion system according to claim 4, wherein the first anchor and the second anchor are arranged in the expanded configuration such that their respective lobes are offset with respect to each other.

9. The bioabsorbable occlusion system according to claim 1, wherein the first anchor and the second anchor in the expanded configuration are substantially disc shaped.

10. The bioabsorbable occlusion system according to claim 1, wherein the occlusion implant is substantially bioabsorbable:
between 6 months and 18 months from implantation;
between 9 months and 18 months from implantation; or
between 12 months and 18 months from implantation.

11. The bioabsorbable occlusion system according to claim 1, wherein an outer edge of the first anchor is at least partially inflatable to define a shape of the first anchor.

12. The bioabsorbable occlusion system according to claim 11, wherein an outer edge of the second anchor is at least partially inflatable to define a shape of the second anchor.

13. The bioabsorbable occlusion system according to claim 1, wherein the first anchor, the second anchor, and the connector element are formed from a substantially bioabsorbable membrane such that an entirety of the occlusion implant is inflatable to attain the expanded configuration.

14. The bioabsorbable occlusion system according to claim 13, wherein the first anchor and the second anchor each have a cross-sectional dimension that is substantially greater than a cross-sectional dimension of the connector element.

15. The bioabsorbable occlusion system according to claim 1, wherein the first anchor and the second anchor each have a cross-sectional dimension that is substantially greater than a cross-sectional dimension of the connector element.

16. The bioabsorbable occlusion system according to claim 1, wherein the first anchor and the second anchor are formed from a frame of anchor members that are entirely absorbable.

17. The bioabsorbable occlusion system according to claim 1, further comprising a guide catheter having a channel that extends therethrough and that carries the first anchor and a second anchor in the insertion configuration.

18. The bioabsorbable occlusion system according to claim 16, further comprising a delivery device movable within and relative to the guide catheter, the delivery device configured to advance the occlusion implant from inside the channel to a location outside the channel, wherein the delivery device is releasably coupled to the occlusion implant.

19. The bioabsorbable occlusion system according to claim 17, wherein the delivery device is an elongated shaft.

20. The bioabsorbable occlusion system according to claim 17, wherein the delivery device is an elongated tube having a channel for receiving a portion of the connector element therein.

21. The bioabsorbable occlusion system according to claim 17, further comprising an access sheath having a proximal end, a distal end, and an access sheath channel that extends from the proximal end to the distal end, wherein the guide catheter is insertable into the access sheath channel.

22. The bioabsorbable occlusion system according to claim 1, wherein the connector element includes a plurality of teeth that are configured to engage a plurality of teeth located in a bore of the first anchor and the second anchor.

23. The bioabsorbable occlusion system according to claim 1, wherein the connector element includes an elongate shaft with a plurality of engagement members that are configured to releasable couple to a plurality of corresponding engagement members in a bore of the first anchor or the second anchor.

24. A method for occluding an opening or defect in a heart, comprising:
inserting a guide catheter over a guidewire so that its distal end is located proximate the opening or defect in the tissue of the heart;
advancing a delivery device in a distal direction through a channel of the guide catheter so that a first anchor and a connector element of an occlusion implant is positioned adjacent to a first side of the tissue adjacent the opening or defect, wherein an entirety of the occlusion implant is substantially bioabsorbable;
further advancing the delivery device in the distal direction through the channel of the guide catheter so that a second anchor of the occlusion implant is positioned adjacent to a second side of the tissue of the heart and overlies the opening or defect;
retracting the connector element to draw the first anchor toward the second anchor to secure occlusion implant in place; and
allowing the occlusion implant to entirely bioabsorb.

25. The method of claim 24, further comprising locking the first and second anchor in place relative to each other to secure the occlusion implant in place.

26. The method of claim 24, wherein the opening or defect is an atrial septal defect.

27. The method of claim 24, wherein the opening or defect is a patent foramen ovale.

28. The method of claim 24, wherein the opening or defect is a result of ductus arteriosis.

29. The method of claim 24, wherein the opening or defect is iatrogenic transcaval defect.

30. The method of claim 24, wherein the opening or defect is a ventricular septal defect.

31. The method of claim 24, wherein the opening or defect is an arterio-venous fistulae.
